(19) United States
(12) Patent Application Publication
(10) Pub. No.: US 2024/0369547 A1
(43) Pub. Date: Nov. 7, 2024

(54) SYSTEMS AND METHODS FOR PATHOGEN DETECTION

(71) Applicant: University of Maryland, College Park, College Park, MD (US)

(72) Inventors: Kevin M. DANIELS, Columbia, MD (US); Soaram KIM, Glen Burnie, MD (US); John Robertson RZASA, University Park, MD (US)

(21) Appl. No.: 18/576,439

(22) PCT Filed: Jul. 20, 2022
(86) PCT No.: PCT/US2022/037666

§ 371 Date: Jan. 4, 2024

Related U.S. Application Data
(60) Provisional application No. 63/223,919, filed on Jul. 20, 2021.

(51) Int. Cl. G01N 33/543; G01N 33/569

(52) U.S. Cl. CPC G01N 33/5438; G01N 33/56983; G01N 2333/165; G01N 2469/10

(57) ABSTRACT

A system for the sensing of pathogens includes a sensor, a processor, and a memory. The sensor is configured to generate a signal indicating a conductivity of the sensor. The memory includes instructions stored thereon, which, when executed by the processor, cause the system to receive the signal from the sensor, determine a change in a conductivity of the sensor based on the signal, and determine the presence or the absence of a pathogen based on the determined change in conductivity.

### CROSS-REFERENCE TO RELATED APPLICATION/CLAIM OF PRIORITY

This is a national stage application filed under 37 U.S.C. 371 based on International Patent Application No. PCT/US2022/037666, filed Jul. 20, 2022, which claims the benefit of, and priority to, U.S. Provisional Patent Application No. 63/223,919 filed on Jul. 20, 2021, the entire contents of which are hereby incorporated by reference.

### TECHNICAL FIELD

The present disclosure relates generally to the field of pathogen detection. More specifically, an aspect of the present disclosure provides devices, systems, and methods for wireless sensing of pathogens.

### BACKGROUND

SARS-CoV-2, the virus responsible for COVID-19, can be highly infectious in recently infected individuals and in asymptomatic carriers, which has led to the global spread of COVID-19. Remdesivir, the first treatment for COVID-19, has shown a prominent effect on inhibiting the early life cycle of coronavirus replication. Monoclonal antibodies, which target the spike protein of SARS-CoV-2, are another effective treatment for preventing death in high-risk COVID-19 patients. Vaccines, including BNT162b2, mRNA-1273, JNJ-78436735, AZD1222, and NVX-CoV2373, have been shown to prevent infection or reduce the symptoms of COVID-19. However, the reduced effectiveness of the vaccines and monoclonal antibodies in preventing severe illness from variants such as B.1.1.7, B.1.351, B.1.429, B.1.526, B.1.617, and P.1 remains a concern. In addition, the global rollout of the vaccines will take time. The key to preventing the spread of COVID-19 is early detection before developing symptoms. Hence, ultrafast, highly sensitive diagnostic methods are needed to prevent the further spread of COVID-19. Real-time reverse transcription-polymerase chain reaction (RT-PCR), the gold standard for COVID-19 diagnostics, detects infection through the genetic makeup of SARS-CoV-2. However, due to its sophisticated and time-consuming experimental settings, rapid, on-site diagnosis is not feasible. Accordingly, there is interest in an improved means for pathogen detection.

### SUMMARY

An aspect of the present disclosure provides a system for the detecting of pathogens. The system includes a sensor, a processor, and a memory. The sensor is configured to generate a signal indicating a conductivity of the sensor. The memory includes instructions stored thereon, which, when executed by the processor, cause the system to receive a signal from the sensor, determine a change in a conductivity of the sensor based on the signal, and determine the presence or the absence of a pathogen based on the determined change in conductivity.

In an aspect of the present disclosure, the sensor may include quasi-freestanding bilayer epitaxial graphene.

In yet another aspect of the present disclosure, the instructions, when executed by the processor, may further cause the system to compare the signal to a pre-determined conductivity value.

In another aspect of the present disclosure, the system may further include a display. The instructions, when executed by the processor, may further cause the system to display the determined presence or absence of the pathogen.

In yet another aspect of the present disclosure, the sensor may include a first layer of a non-conductive material, a second layer of a conductive material having an antibody bonded thereto, and a set of electrodes disposed on the second layer.

In another aspect of the present disclosure, the antibody may be bonded to the conductive material via a crosslinker.

In yet another aspect of the present disclosure, the set of electrodes may be configured to supply a charge to the conductive material in the sensor.

In another aspect of the present disclosure, the antibody may correspond to an antigen to be sensed.

In yet another aspect of the present disclosure, the sensor may be reusable.

In another aspect, the instructions, when executed by the processor, may further cause the system to transmit an indication of the determined presence or absence of the pathogen to a user device.

An aspect of the present disclosure provides a sensor that may include a first layer including a non-conductive material, a second layer including a conductive material having an antibody bonded thereto, and a set of electrodes disposed on the second layer.

In another aspect of the present disclosure, the first layer and the second layer may be formed using thermal decomposition.

In yet another aspect of the present disclosure, the conductive material may be quasi-freestanding bilayer epitaxial graphene.

In another aspect of the present disclosure, the set of electrodes may be configured to supply a charge to the conductive material.

In yet another aspect of the present disclosure, the antibody may correspond to an antigen to be sensed.

In another aspect of the present disclosure, the antibody may be chemically bonded to the conductive material via a crosslinker.

In another aspect of the present disclosure, the sensor may be configured to generate a signal indicating a conductivity of the sensor.

An aspect of the present disclosure provides a computer-implemented method for pathogen detection. The computer-implemented method may include determining a change in conductivity of a graphene-based sensor and determining the presence and/or the absence of a pathogen based on the determined change in conductivity.

In another aspect of the present disclosure, the computer-implemented method may further include supplying a charge to a conductive material of the graphene-based sensor using a set of electrodes.

In another aspect, the computer-implemented method may further include displaying on a user device the determined presence of the pathogen.

Further details and aspects of the present disclosure are described in more detail below with reference to the appended drawings.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying drawings of which:

FIG. 1 is a diagram of a system for detecting a pathogen, in accordance with aspects of the disclosure;

FIG. 2 is a block diagram of a controller configured for use with the system for pathogen detection of FIG. 1, in accordance with aspects of the disclosure;

FIG. 3 is a diagram of a sensor for detecting an antigen, in accordance with aspects of the present disclosure;

FIGS. 4A-D are diagrams illustrating components of the sensor of FIG. 3, in accordance with aspects of the present disclosure;

FIGS. 5A-B are atomic force microscopy graphs illustrating differences in a composition of the sensor of FIG. 3. before and after contact with the antigen, in accordance with aspects of the disclosure;

FIG. 6 is a block diagram showing the components of the sensor of FIG. 3 and an exemplary method of making it, including the steps of thermal decomposition and covalent bonding to a spike protein of an antibody corresponding to the antigen, in accordance with aspects of the disclosure;

FIG. 7 is a graph illustrating the sensitivity and speed of the sensor of FIG. 3 in the presence of the antigen in various concentrations with clear changes in electrical conductivity, in accordance with aspects of the disclosure;

FIG. 8 is a graph illustrating the effectiveness of the sensor in response to several antigen variants of a pathogen, in accordance with aspects of the present disclosure;

FIGS. 9A-C are graphs illustrating the effectiveness of the sensor of FIG. 3 with varying forms of samples introduced, in accordance with aspects of the disclosure; and

FIG. 10 is a flow diagram for a computer-implemented method of pathogen detection in accordance with aspects of the disclosure.

### DETAILED DESCRIPTION

The present disclosure relates generally to the field of pathogen detection. More specifically, an aspect of the present disclosure provides devices, systems, and methods for wireless sensing of antigens.

Aspects of the present disclosure are described in detail with reference to the drawings wherein like reference numerals identify similar or identical elements.

Although the present disclosure will be described in terms of specific aspects and examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary aspects illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

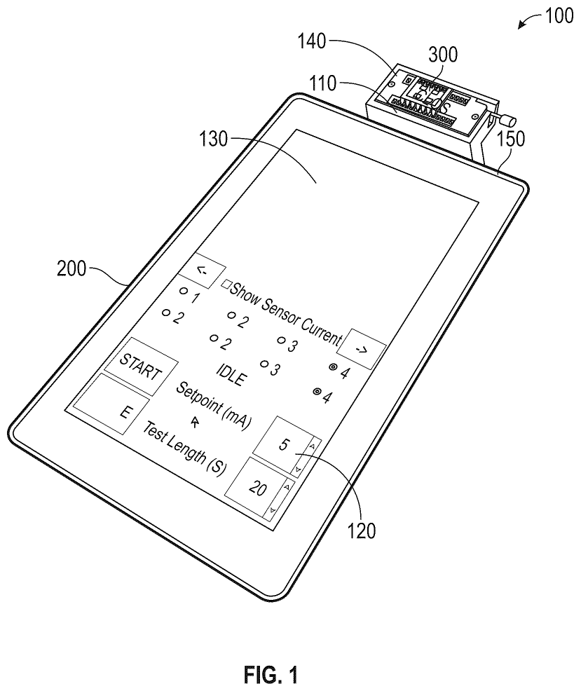

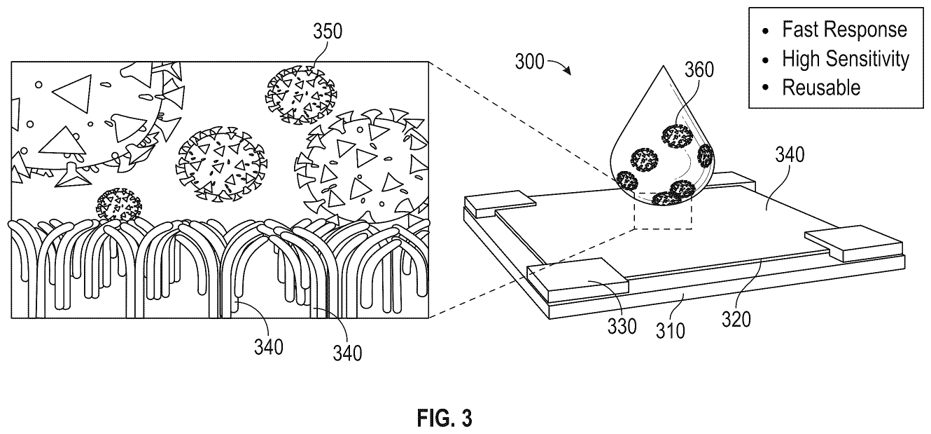

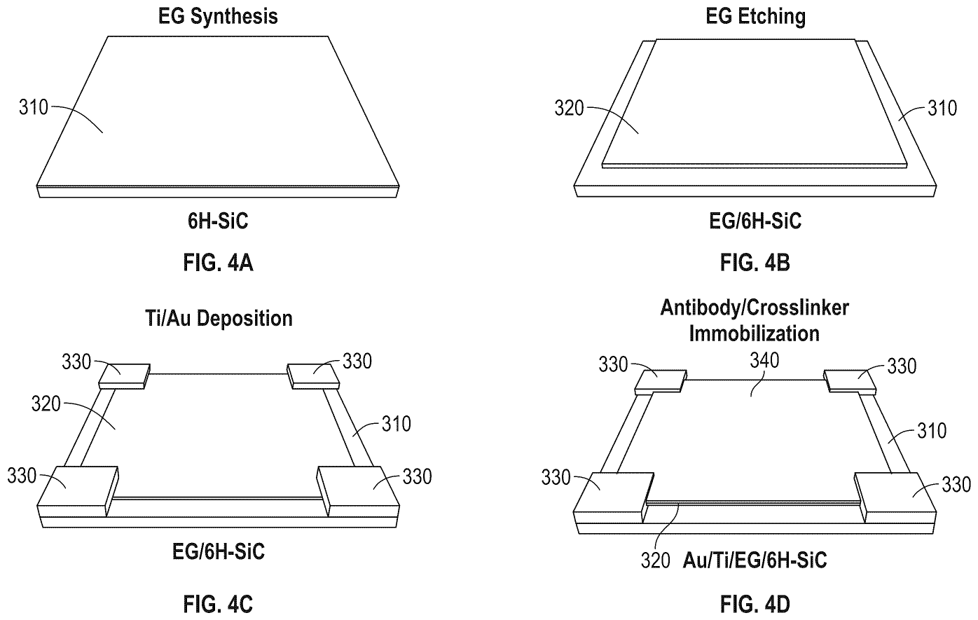

Referring to FIG. 1, a system 100 for detecting the presence or absence of a pathogen is shown. The system 100 may be configured to sense whether a pathogen is present by sensing for an antigen 350 (FIG. 3) of the pathogen. In aspects, the pathogen may be the COVID-19 virus. The antigen may be a surface protein of a virus. The system 100 generally includes a controller 200 and/or a sensor 300 configured to receive a sample 360. The sensor 300 may be connected to the controller 200 by a connector 110. The sensor 300 may be a quasi-freestanding (“QFS”) bilayer epitaxial graphene (“EG”) based sensor. Graphene is a single atomic layer of carbon atoms with a relatively high surface area. Graphene has shown exceptionally high sensitivity, less than about 1 part per billion, with high electrical conductivity and carrier mobility (e.g., about 100,000 cm²/Vs theoretically). QFS bilayer EG has several advantages, such as improved thickness uniformity, reduced phonon-carrier scattering, and/or higher mobility by hydrogen intercalation compared to conventional EG. In aspects, the sensor 300 may contain four parts: a semi-insulating substrate (e.g., SiC), QFS bilayer EG, a crosslinker with an antibody corresponding to an antigen, and electrodes (FIGS. 4A-D). The high quality and uniformity of EG enable the bottom-up direct immobilization of both crosslinker and the spike protein antibodies on EG without any complex transfer methods. EG supports large area synthesis (e.g., up to wafer size range) for commercial scale-up.

The system 100 may include a voltage and/or current source 140 configured to generate a voltage and/or current to be applied across the sensor 300. The system 100 may further include a second sensor 150 (e.g., high speed galvanostat circuit, a voltage sensor, and/or a current sensor) configured to measure a conductivity of the sensor 300 (e.g., the graphene sensor) and generate a signal reflecting the conductivity. The system 100 may include a display 130 and/or a touch screen 120. The touch screen 120 is configured to enable a user to view testing results of the system 100. The touch screen 120 may be further configured for enabling a user to assign, for example, a setpoint, a start, and/or a test length. The system 100 may be used as a pathogen sensor, which may utilize the conductivity of the sensor 300 to determine the presence or absence of an antigen 350 of a pathogen within a sample. In aspects, the sample may be as small as one copy of the virus.

In aspects, the system 100 may be configured to send test results to other devices and systems, such as electronic medical records. In aspects, the test results may be sent through wireless communication or similar means. The system 100 may enable the user to set a start point, a setpoint, and/or a test time length. The system 100 may be portable to enable convenient, accurate, and/or widespread testing capabilities.

Although COVID-19 virus variants are discussed, the disclosed technology may be used to determine the presence or absence of any pathogen with a known associated antigen to which an antibody exists or may be developed.

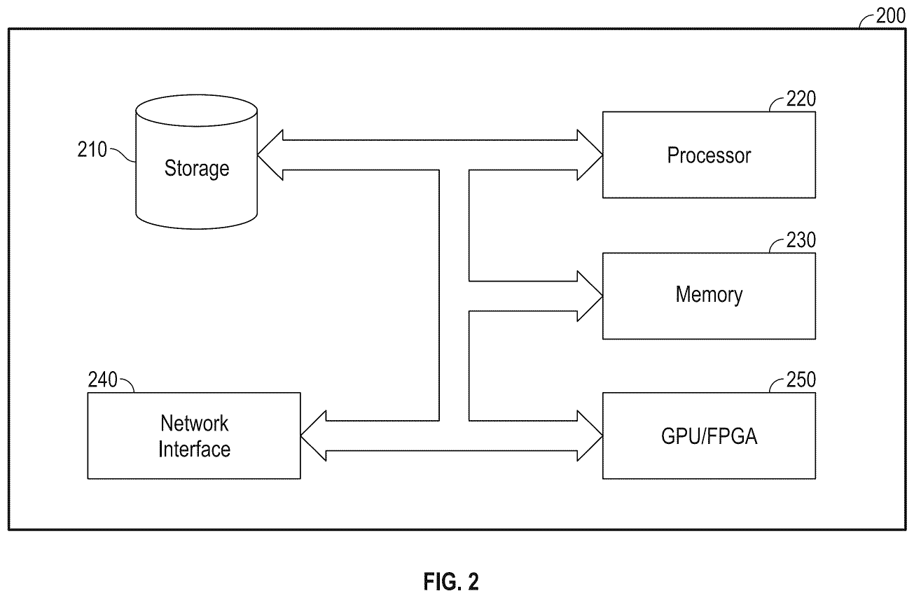

Referring to FIG. 2 controller 200 includes a processor 220 connected to a computer-readable storage medium or a memory 230. The controller 200 may be used to control and/or execute operations of the system 100, including the configuration of display 130 and/or touch screen 120. The computer-readable storage medium or memory 230 may be a volatile type of memory, e.g., RAM, and/or a non-volatile type of memory, e.g., flash media, disk media, etc. In various aspects of the disclosure, the processor 220 may be another type of processor, such as a digital signal processor, a microprocessor, an ASIC, a graphics processing unit (GPU), a field-programmable gate array (FPGA), and/or a central processing unit (CPU). Further, processor 220 generally may refer to one or more or any such processing devices. In certain aspects of the disclosure, network inference may also be accomplished in systems that have weights implemented as memristors, chemically, or other inference calculations, as opposed to processors.

In aspects, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 may include computer-readable instructions that are executable by the processor 220 to operate the controller 200. In aspects of the disclosure, the controller 200 may include a network interface 240 to communicate with other computers or a server. A storage device 210 may be used for storing data. The disclosed method may run on the controller 200 and/or on a user device, including, for example, on a mobile device, an IoT device, or a server system.

With reference to FIG. 3, an illustration of the sensor 300 interacting with a sample 360 is shown. The sensor 300 generally includes a semi-insulating non-conductive layer 310. The semi-insulating non-conductive layer 310 may include silicon carbide on which a conductive EG layer 320 is disposed. The conductive EG layer 320 may be disposed on the semi-insulating non-conductive layer 310 through a thermal decomposition process. Two or more electrodes 330 may be disposed on the conductive EG layer 320. Further, an antibody 340 associated with an antigen of the pathogen (that may be detected based on the presence or absence of an antigen 350 in the sample 360) may be disposed on the conductive EG layer 320. The sensor 300 may be configured for detecting the presence or absence of the antigen 350 in real-time based on a change in the conductivity of the sensor 300.

Referring to FIGS. 4A-D, diagrams illustrating the synthesis and layering of the parts of the sensor 300 are shown. In FIG. 4A, the semi-insulating non-conductive layer 310 is formed on a base layer of non-conductive material. The non-conductive material may be SiC.

FIG. 4B shows a conductive EG layer 320 disposed on the semi-insulating non-conductive layer 310 with an etching process. The etching may be performed on an edge side on EG using CF₄ plasma with a simple mask. For example, high-quality EG may be synthesized through Si sublimation and subsequent hydrogen intercalation on about a 4-inch diameter semi-insulating (0001) ˜0.1° off-axis 6H-SiC using a horizontal hot-wall reactor. After dicing the EG/SiC samples (8 mm×8 mm), the EG/SiC samples may be cleaned with acetone and isopropanol (IPA), rinsed with deionized (DI) water, and/or dried with pure N₂. A mesa may be etched on the EG samples by CF4 plasma with a simple mask (about 7 mm×about 7 mm) to isolate the EG on SiC.

FIG. 4C shows the deposition of a set of electrodes 330 disposed on the conductive EG layer 320. The electrodes 330 may provide a flow of electricity to the sensor 300. The electrodes 330 may be made of any suitable material. For example, a titanium gold (Ti/Au) metal stack may be used for the electrodes 330. The formation of the mesa may be followed by the deposition of the four contact electrodes (2 mm×2 mm), Ti/Au (30 nm/220 nm), by electron beam evaporation on EG.

FIG. 4D shows immobilization of an antibody 340 corresponding to the antigen 350 to be detected, on the conductive EG layer 320. The bonding may include using a crosslinker for immobilization of the antibody 340. In an aspect, the sensor 300 may use polyclonal antibodies for the antibody EG heterostructure with the EG layer. The sensor 300 may recognize different epitopes on the same protein antigen, permitting a low limit of detection. For example, the crosslinker, 0.1% of poly L lysine, may be diluted in DI water to 0.01%, and the crosslinker and S1 antibodies then immobilized on EG. The crosslinker may be immobilized on the EG by contacting a solution of the crosslinker with the EG for about one hour at room temperature. Then, the S1 protein antibodies may be immobilized onto the crosslinker by adsorption due to hydrophobic interaction between antibody and crosslinker (similar to immunoassay coating process) by exposing the crosslinker to the antibody 340 for about 12 hours at about 4° C. after diluting-e.g., the SARS-CoV-2 spike S1 protein (about 1:1000 in ELISA coating buffer (1×)).

In aspects, the conductive EG layer 320 may include high sensitivity QFS bilayer EG, which may then be etched. The high quality and uniformity of the EG may permit the bottom-up direct immobilization of both the crosslinker and the antibody on the EG without complex transfer methods. The antibody 340 has high selectivity and may be incorporated onto the conductive EG layer 320 through a crosslinker and an immobilization procedure to ensure stable bonding.

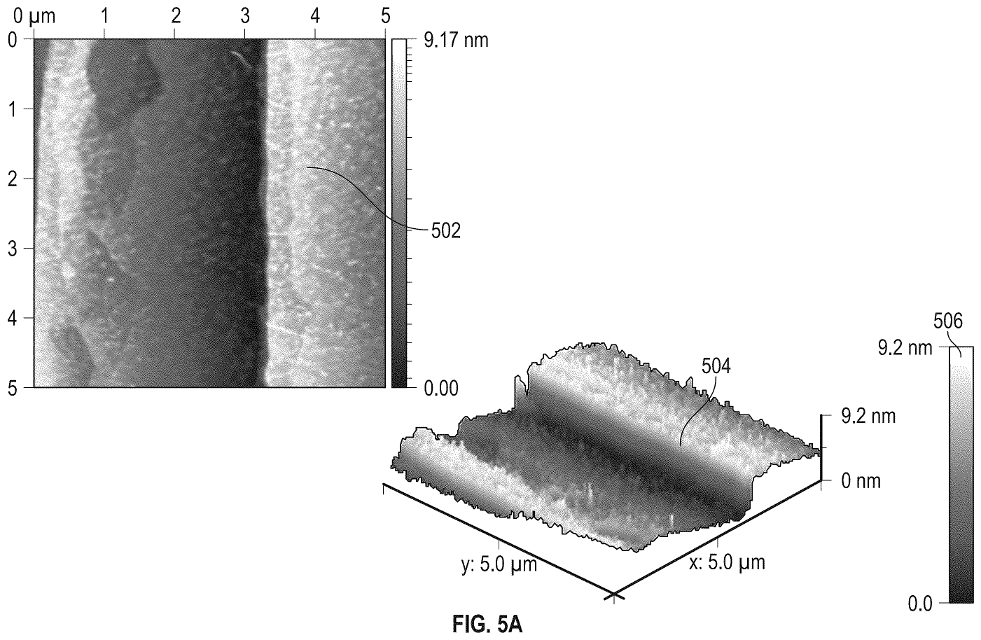

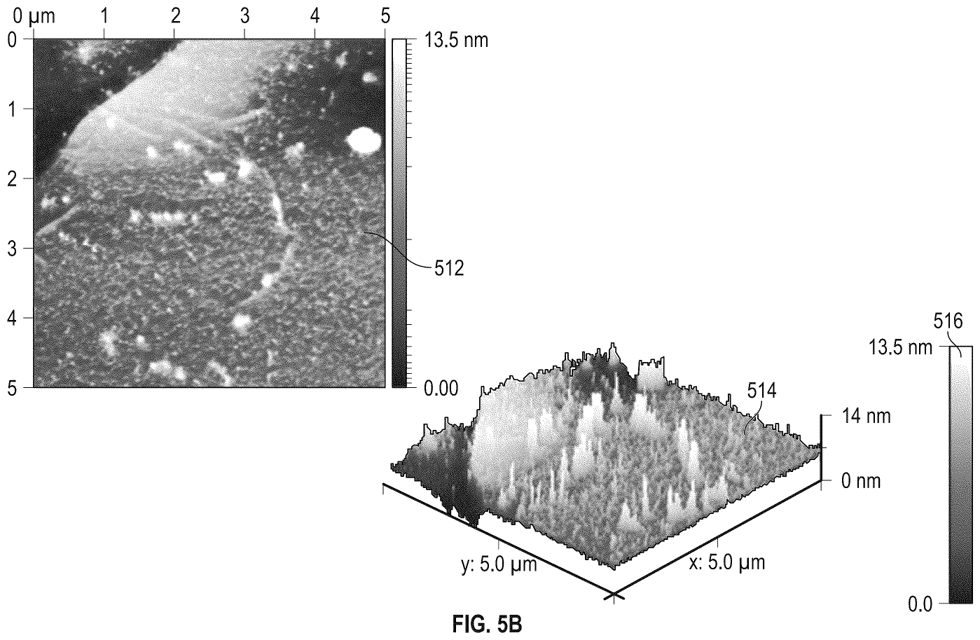

Referring to FIG. 5A and 5B, atomic force microscopy of a mesa made of EG is shown. FIG. 5A shows the QFS bilayer EG on SiC before the addition of the antibody/crosslinker is added. The root-mean-square (RMS) roughness of the crosslinker and the QFS bilayer EG in FIG. 5A may be about 0.8 nm and about 0.7 nm, respectively. Graph 504 is a 3D view of graph 502. Scale 506 indicates the structural range of graphs 502 and 504.

FIG. 5B illustrates the atomic force microscope images of SARS-CoV-2 S1 spike protein antibody/crosslinker prepared on EG/SiC. The immobilized antibody/crosslinker may be uniform and dense on EG, indicating RMS roughness of about 1.9 nm. The change between FIG. 5A and FIG. 5B demonstrates the change in the mesa following crosslinker on the EG surface, which bonds the antibody 340 (FIG. 3) to the sensor 300 (FIG. 1) surface. Graph 512 is a 3D view of graph 514. Scale 516 indicates the structural range of graphs 512 and 514.

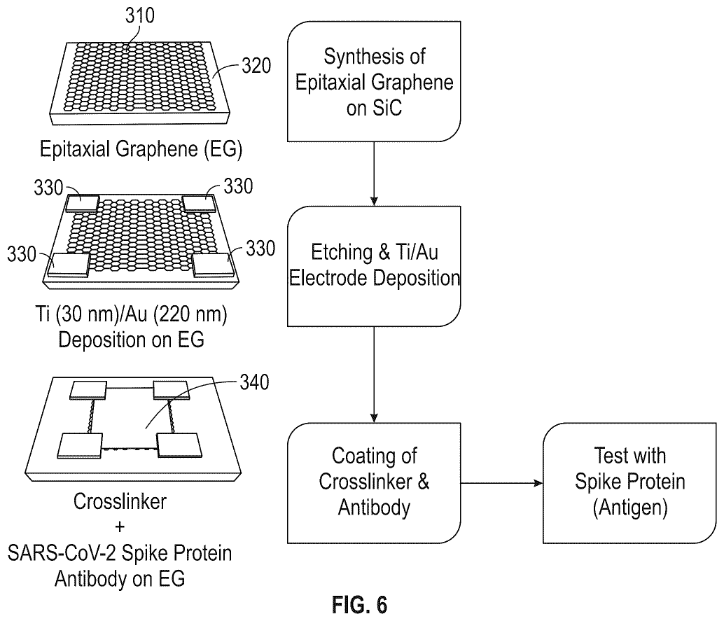

FIG. 6 illustrates the stepwise configuration of the sensor 300. QFS bilayer EG may be synthesized on a semiconductor layer of SiC (e.g., semi-insulating non-conductive layer 310) through thermal decomposition. The set of electrodes 330 may include Ti/Au alloy. The electrodes 330 may be applied to the EG layer to deliver a charge generated by voltage and/or current source 140 (FIG. 1). A coating of crosslinker and a spike protein of an antibody 340 corresponding to the antigen 350 to be detected may be applied to the EG layer to bond to the graphene surface.

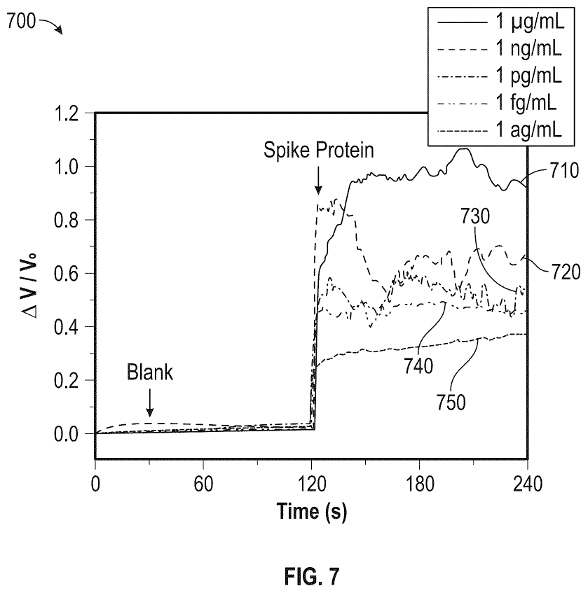

FIG. 7 illustrates the sensitivity of the sensor 300 (FIG. 1) at varied antigen 350 (FIG. 3) concentrations from about one attogram (10⁻¹⁸ grams) to about one microgram (10⁻⁶ grams) of the antigen 350 in the sample 360. In aspects, the sensor 300 may respond to as low as one attogram of the antigen 350. The sensor 300 may be highly accurate and may provide a sensitivity that is significantly lower than the limit of detection for conventional immunoassays and other biosensors.

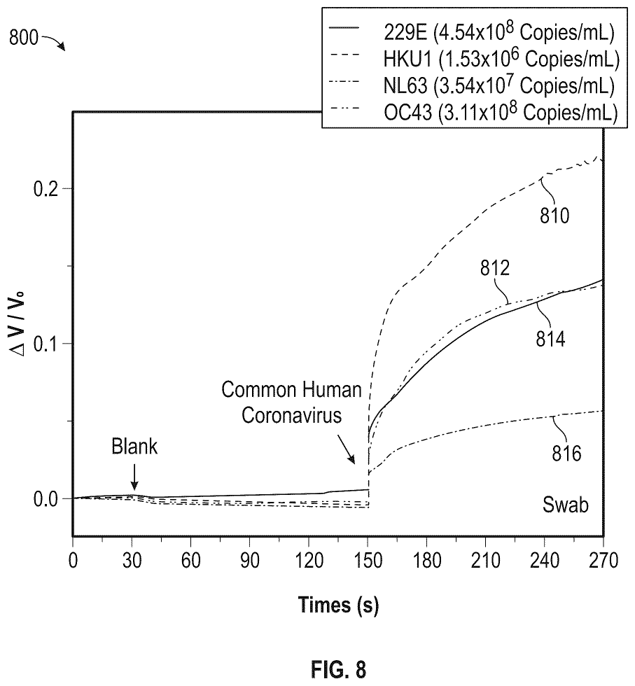

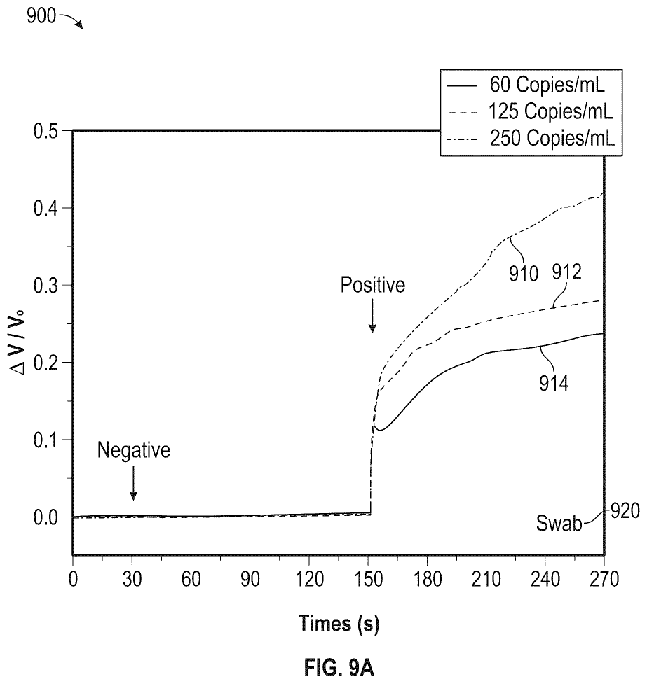

With reference to FIG. 8, the sensitivity of the sensor 300 (FIG. 1) when introduced to several variants of the same pathogen within a sample 360 (FIG. 3) is illustrated. In aspects, the sensor 300 may detect four common human coronavirus variants, including 229E 814, HKU1 810, NL63 816, and/or OC43 812. In aspects, the sensor 300 may provide discernable results in less than two minutes.

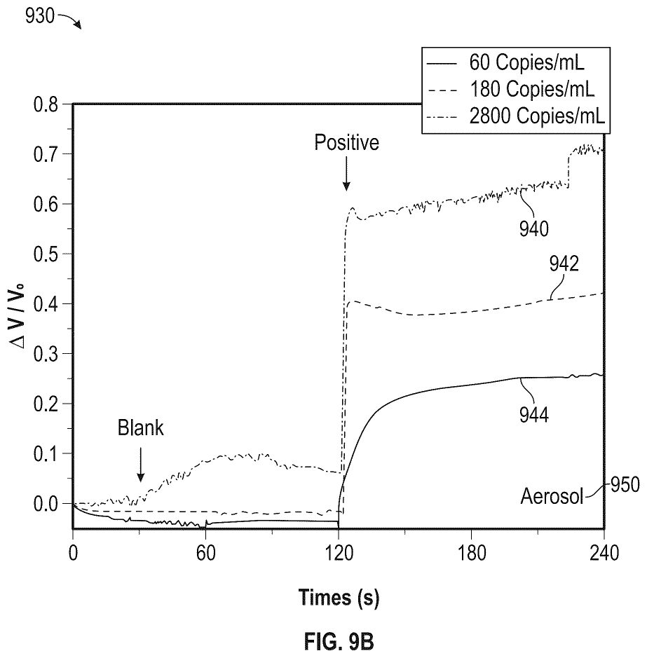

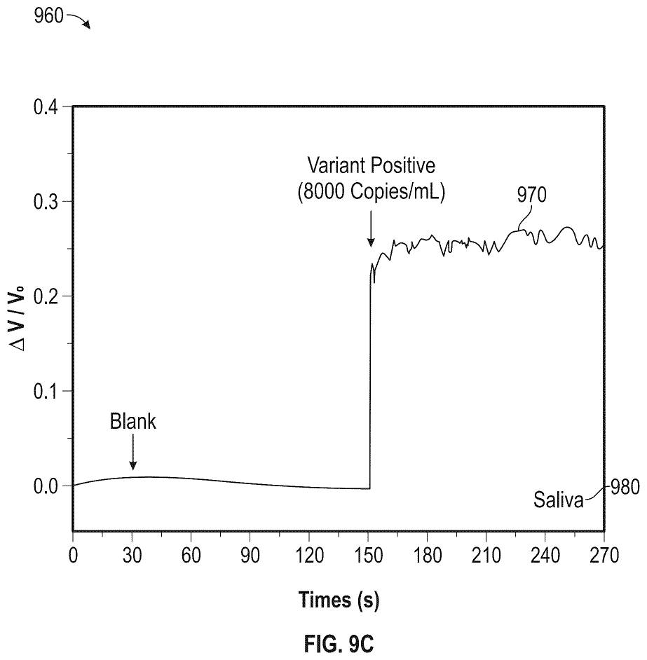

With reference to FIGS. 9A-C, the sensitivity of the sensor 300 (FIG. 1) when introduced to varied forms of sample 360 (FIG. 3) is illustrated. In aspects, the sensor 300 may function with accuracy and speed with a variety of sample types, including mid-turbinate swabs 920, saliva 980, and/or aerosol 950 samples. For each sample type, the sensor 300 may clearly distinguish between pathogen-negative and pathogen-positive samples in seconds.

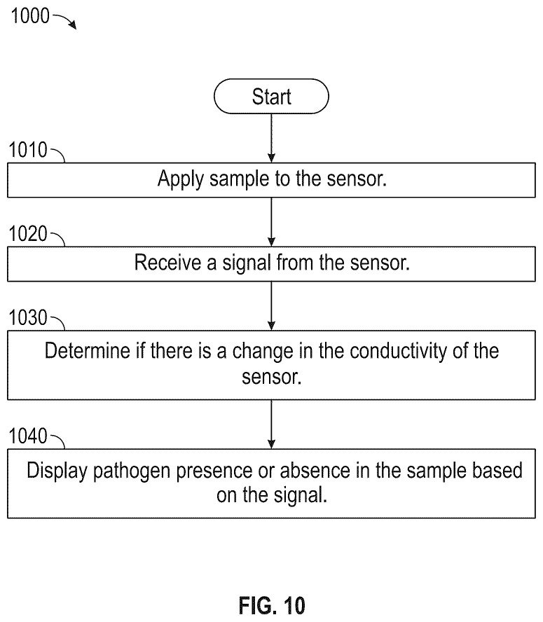

Referring to FIG. 10, a flow diagram for a method in accordance with the present disclosure for detecting a pathogen is shown as 1000. Although the steps of FIG. 10 are shown in a particular order, the steps need not all be performed in the specified order, and certain steps can be performed in another order. For example, FIG. 10 will be described below, with a controller 200 of FIG. 2 performing the operations. In aspects, the operations of FIG. 10 may be performed all or in part by another device, for example, a server, a mobile device, such as a smartphone, and/or a computer system. These variations are contemplated to be within the scope of the present disclosure.

Initially, at step 1010, the sample 360 (FIG. 3) in either mid-turbinate swab 920 (FIG. 9A), saliva 980 (FIG. 9C), aerosol 950 (FIG. 9B), and/or other form is applied to the conductive EG layer 320 (i.e., heterostructure surface including antibody 340, see FIG. 3) of sensor 300 (FIG. 1). For example, saliva samples may be stored as is, or may be diluted with 1× Phosphate-buffered saline containing about 0.1% bovine serum albumin (PBS/BSA) when the volume is less than about 1 mL. Although one sensor is used as an example, multiple sensors may be tested at once, for example, using one breath. In aspects, strain induced on the QFS EG may create a G-peak shift, enabling the transduction of antibody-antigen bindings. For example, a pathogen may be detected based on electrical transduction of the SARS-CoV-2 S1 spike protein antigen via SARS-CoV-2 S1 spike protein antibodies immobilized on QFS EG. This polarization-induced strain enables the electrical transduction of as little as about 1 attogram per millimeter of the SARS-CoV-2 S1 spike protein antigen upon binding with the SARS-CoV-2 S1 spike protein antibodies immobilized on QFS EG. These polarization fields have the benefit of enabling the electrical transduction of bindings through a sensor design that does not require the typical field effect transistor architecture.

The conductivity of the sensor 300 may be configured to change based on the presence of an antigen 350 (FIG. 3) of a particular pathogen. In aspects, the controller 200 may supply a charge to the conductive EG layer 320 in the sensor 300 using the set of electrodes 330 (FIG. 3). The charge may be generated by voltage and/or current source 140 (FIG. 1). In the presence of the antigen 350, the conductivity of the sensor 300 may increase. For example, an input current of 10 mA may be applied to the sensor 300 directly and maintained during the measurement. The detected output electrical response may be normalized as ΔV/Vₒ, where ΔV is the change in voltage and Vₒ is the original voltage.

Next, at step 1020, the controller 200 receives a signal from the sensor 300. The signal may indicate a conductivity of the sensor 300. For example, the signal may be a voltage, e.g., about 5 volts. The second sensor 150 may detect the voltage and the controller 200 may compare the voltage to a stored calibration voltage for the sensor 300. For example, the stored calibration voltage may be about 2 volts. The controller 200 may compare the sensed 5 volts to the stored calibration voltage of about 2 volts and determine the presence of the antigen 350 based on the difference. In aspects, the second sensor 150 may include a high speed galvanostat circuit. The high speed galvanostat circuit may be configured to maintain a constant current through the sensor 300, and then when the resistance of the sensor changes due to interactions with the virus, the galvanostat has to change its compliance voltage to maintain the same current. The signal may indicate a conductivity of the sensor 300 may include the changed compliance voltage.

Next, at step 1030, the controller 200 determines whether there is a change in conductivity of the sensor 300. For example, prior to the sample being placed on the sensor, the sensor may have a conductivity of about 0.0 ΔV/Vₒ, where ΔV is the change in voltage and Vₒ is the original voltage. After a positive sample is introduced, the conductivity may change to about 0.5 ΔV/Vₒ. which is over the threshold of about 0.05 ΔV/Vₒ. The controller 200 may process the signal to determine if the antigen 350 is absent or present in response to the change in conductivity of the sensor 300. The controller 200 may determine that the change in conductivity of the sensor 300 indicates the absence or presence of an antigen 350 of a pathogen being sensed. The controller 200 may use a voltage sensor to sense the change in conductivity of the sensor 300. The controller 200 may compare the signal to a pre-determined threshold value. For example, a significant change in conductivity may indicate the antigen 350 is present and the sample is positive. For example, no change in conductivity or an insignificant change in conductivity may indicate the pathogen is absent, and the sample is negative. Based on the presence of the antigen 350, the controller 200 may determine that a pathogen is present. For example, the spike protein of an antibody 340 may be associated with a common human coronavirus SARS-CoV-2 antigen, which is responsible for the COVID-19 virus. When a sample containing SARS-CoV-2 is introduced to the sensor 300, the ΔV/Vₒ may indicate the presence of the SARS-CoV-2 by demonstrating an increase in conductivity with a higher voltage passing through the EG.

Next, at step 1040, the controller 200 displays whether the pathogen is present or absent. For example, the display 130 (FIG. 1) may display that the pathogen is present in the sample by showing an increase in voltage flowing through the conductive EG layer 320 of sensor 300. In another example, the sensor 300 may be configured to sense SARS-CoV-2, the antigen 350 responsible for the COVID-19 pathogen. The antibody EG heterostructure may consist of an antibody 340 associated with SARS-CoV-2, which may change the EG configuration to be more conductive and to pass more voltage through the conductive EG layer 320 when a SARS-CoV-2 antigen is present in the sample. The sensor 300 may provide the benefit of real-time ultra-sensitive sensing of pathogens.

In aspects, the sensor 300 may be reusable. The sensor 300 may be cleaned and reused by heating the sensor 300 to a temperature greater than about 40° C. The sensor 300 may also be cleaned for reuse by soaking the sensor 300 in a salt solution.

In aspects, the controller 200 is configured to wirelessly transmit (e.g., by Bluetooth™ or other wireless protocol) the data to a smartphone application. In aspects, the data may be wirelessly transmitted (securely) to other types of authorized systems/devices, including but not limited to local health monitoring devices, remote health monitoring systems (e.g., cloud-based and perhaps operated by a healthcare provider), and/or a combination of a local health monitoring device that provides health monitoring information to a health monitoring system.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. For example, while described with respect to an antibody to an antigen on the pathogen, it should be understood that any specific binding pair (e.g., not involving antibodies) may be used for detection in the present systems. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for detecting a pathogen, the system comprising:
a sensor configured to generate a signal indicating a conductivity of the sensor;
a processor; and
a memory including instructions stored thereon, which, when executed by the processor, cause the system to:
receive the signal from the sensor;
determine a change in a conductivity of the sensor based on the signal; and
determine the presence or the absence of a pathogen based on the determined change in conductivity.

2. The system of claim 1, wherein the sensor includes quasi-freestanding bilayer epitaxial graphene.

3. The system of claim 1, wherein the instructions, when executed by the processor, further cause the system to compare the signal to a pre-determined threshold conductivity value.

4. The system of claim 1, the system further including a display, and
wherein the instructions, when executed by the processor, further cause the system to display the determined presence or absence of the pathogen.

5. The system of claim 1, wherein the sensor includes:
a first layer including a non-conductive material;
a second layer including a conductive material having an antibody bonded thereto; and
a set of electrodes disposed on the second layer.

6. The system of claim 5, wherein the antibody is bonded to the conductive material via a crosslinker.

7. The system of claim 5, wherein the set of electrodes is configured to supply a charge to the conductive material in the sensor.

8. The system of claim 5, wherein the antibody corresponds to an antigen to be sensed.

9. The system of claim 5, wherein the sensor is reusable.

10. The system of claim 1, wherein the instructions, when executed by the processor, further cause the system to transmit an indication of the determined presence or absence of the pathogen to a user device.

11. A sensor for detecting an antigen, the sensor comprising:
a first layer including a non-conductive material;
a second layer including a conductive material having an antibody bonded thereto; and
a set of electrodes disposed on the second layer.

12. The sensor of claim 11, wherein the first layer and second layer are formed using thermal decomposition.

13. The sensor of claim 11, wherein the conductive material includes quasi-freestanding bilayer epitaxial graphene.

14. The sensor of claim 11, wherein the set of electrodes is configured to supply a charge to the conductive material.

15. The sensor of claim 11, wherein the antibody corresponds to an antigen to be sensed.

16. The sensor of claim 11, wherein the antibody is chemically bonded to the conductive material via a crosslinker.

17. The sensor of claim 11, wherein the sensor is configured to generate a signal indicating a conductivity of the sensor.

18. A computer-implemented method for pathogen detection comprising:
determining a change in a conductivity of a graphene-based sensor; and
determining the presence or the absence of a pathogen based on the determined change in conductivity.

19. The computer-implemented method of claim 18, further comprising supplying a charge to a conductive material of the graphene-based sensor using a set of electrodes.

20. The computer-implemented method of claim 18, further comprising displaying on a user device the determined presence of the pathogen.
